(19) United States
(12) Patent Application Publication
(10) Pub. No.: US 2025/0127406 A1
(43) Pub. Date: Apr. 24, 2025

(54) SYSTEM AND METHOD FOR POTASSIUM MEASUREMENT

(71) Applicant: Cirrus Logic International Semiconductor Ltd., Edinburgh (GB)

(72) Inventor: John P. LESSO, Edinburgh (GB)

(73) Assignee: Cirrus Logic International Semiconductor Ltd., Edinburgh (GB)

(21) Appl. No.: 18/912,746

(22) Filed: Oct. 11, 2024

Related U.S. Application Data
(60) Provisional application No. 63/592,295, filed on Oct. 23, 2023.

(51) Int. Cl. A61B 5/0205; A61B 5/00; A61B 5/024; A61B 5/145; A61B 5/1477; A61B 5/33

(52) U.S. Cl. CPC A61B 5/0205; A61B 5/14546; A61B 5/1477; A61B 5/33; A61B 5/6831; A61B 5/7267; A61B 5/7275; A61B 5/746; A61B 5/02416; A61B 5/02438

(57) ABSTRACT

A system for potassium measurement comprising: a chemical sensor configured to output a chemical sensor output signal indicative of a first estimate of a potassium level of a user of the system; a cardiac sensor configured to output a cardiac sensor output signal indicative of a second estimate of the potassium level of the user; and a controller configured to generate an output indicative of the potassium level of the user based on the first and second estimates.

### FIELD OF THE INVENTION

The present disclosure relates to a system and method for potassium measurement.

### BACKGROUND

Potassium is a critical ion in the human body responsible for various physiological processes. Abnormal potassium levels, either hyperkalemia (an elevated potassium level) or hypokalemia (a reduced potassium level), can cause significant health risks. Current potassium measurement methods primarily rely on invasive blood tests which can have multiple sources of error.

### SUMMARY

According to a first aspect, the invention provides a system for potassium measurement comprising: a chemical sensor configured to output a chemical sensor output signal indicative of a first estimate of a potassium level of a user of the system; a cardiac sensor configured to output a cardiac sensor output signal indicative of a second estimate of the potassium level of the user; and a controller configured to generate an output indicative of the potassium level of the user based on the first and second estimates.

The chemical sensor may comprise an ion-selective electrode (ISE) sensor.

The chemical sensor may comprise a potentiometric sensor.

The chemical sensor may comprise a wearable sensor.

The chemical sensor may be configured to analyse a sample of venous blood or to analyse a sample of blood obtained from a fingerprick.

The chemical sensor may comprise an aptamer-based sensor configured to tracks a biomarker associated with potassium levels.

The cardiac sensor may comprise an electrocardiogram (ECG) sensor.

The cardiac sensor may comprise a photoplethysmogram (PPG) sensor.

The system may further comprise an output transducer subsystem.

The output transducer subsystem may comprise one or more of: an audio output transducer; a haptic output transducer; and a visual indicator.

The system may further comprise a communications subsystem configured to communicate data and/or alerts from the system to an external device.

The system may further comprise an output transducer subsystem and a communications subsystem. The controller may be configured to generate an output signal for one or more of output transducer subsystem and the communications subsystem based on the output indicative of the potassium level.

The system may further comprise a predictor configured to generate an estimate of a current and/or future potassium level of the user based on the cardiac sensor output signal.

The predictor may be configured to generate the estimate of the current and/or future potassium level of the user based on the cardiac sensor output signal and the chemical sensor output signal.

The predictor may comprise a trained neural network, artificial intelligence or other machine learning processor.

The predictor may have been trained on a relationship between a potassium level and cardiac data to generate, based on the cardiac sensor output signal, the estimate of the current and/or future potassium level of the user.

The predictor may comprise a trained neural network, artificial intelligence or other machine learning processor. The predictor may have been trained on a relationship between a potassium level and cardiac data and for different levels of potassium to generate, based on the cardiac sensor output signal and the chemical sensor output signal, the estimate of the current and/or future potassium level of the user

According to a second aspect, the invention provides an integrated circuit comprising a controller configured to: receive, from a chemical sensor, a chemical sensor output signal indicative of a first estimate of a potassium level of a user of the system; receive, from a cardiac sensor, a cardiac sensor output signal indicative of a second estimate of the potassium level of the user; and generate an output indicative of the potassium level of the user based on the first and second estimates.

According to a third aspect, the invention provides a method for measuring a potassium level of a subject, the method comprising: generating a first estimate of the potassium level using a chemical sensor; generating a second estimate of the potassium level using a cardiac sensor; and generating an indication of the potassium level based on a fusion of the first and second estimates.

According to a fourth aspect, the invention provides a system for potassium measurement comprising: a controller configured to: receive, from a chemical sensor, a chemical sensor output signal indicative of a first estimate of a potassium level of a user of the system; receive, from a cardiac sensor, a cardiac sensor output signal indicative of a second estimate of the potassium level of the user; and generate an output indicative of the potassium level of the user based on the first and second estimates.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

Embodiments of the invention will now be described, strictly by way of example only, with reference to the accompanying drawings, of which:

FIG. 1 is a schematic representation of an example potassium sensing system according to the present disclosure; and

FIG. 2 is a schematic representation of an alternative example potassium sensing system according to the present disclosure.

### DETAILED DESCRIPTION

Blood test-based potassium measurements can have errors introduced by pseudohyperkalemia. This is a phenomenon where measured potassium levels in a venous blood sample are artificially elevated, not truly reflecting the patient's actual serum potassium level. This condition can arise from a variety of factors. One common cause is mechanical trauma to red blood cells during the blood drawing process, such as when blood is drawn too quickly through a small-bore needle or if the blood sample is excessively shaken. As the cells rupture (hemolysis), they release their intracellular potassium into the serum, leading to a false elevation in the measured potassium level. It is crucial for clinicians to differentiate between true hyperkalemia and pseudohyperkalemia because the management strategies and clinical implications are vastly different. In situations where pseudohyperkalemia is suspected, a repeat blood draw using proper techniques or evaluation of plasma potassium rather than serum potassium may help confirm the actual potassium status.

A wearable potassium sensor would eliminate pseudohyperkalemia. Examples of wearable potassium sensors include Ion Selective Electrode (ISE) sensors having an ion-selective electrode configured to detect potassium as the target ion for the electrode.

However, several sources of error can impact the accuracy and precision of measurements obtained from an ISE. First, the membrane of the ISE, which selectively interacts with the target ion, can deteriorate over time or become contaminated, leading to erroneous readings. Temperature fluctuations can also influence the electrode potential, hence introducing measurement errors if not properly compensated. Interferences from other ions, known as ion interference, can affect the reading, especially if they have similar charge or size to the target ion. Additionally, improper calibration, inadequate sample preparation, or the presence of organic compounds can interfere with the ISE measurement. Ensuring regular maintenance, appropriate calibration, and understanding the specific limitations of the chosen ISE are essential for obtaining accurate results.

Recent studies have shown correlations between potassium levels and certain cardiac signals, such as electrocardiograms (known as ECG or EKG) or photoplethysmograms (PPG).

In an electrocardiogram (ECG or EKG), a component of the monitored signal known as the T-wave represents the repolarization of the ventricles of the heart. The morphology and amplitude of the T-wave can be altered (i.e. may differ from those of a normal T-wave) in the presence of electrolyte imbalances, particularly those involving potassium.

In hyperkalemia (an elevated blood potassium level), the T-wave often becomes tall, peaked, and narrow. As the hyperkalemia becomes more severe, further ECG changes can manifest, such as a widening of the QRS complex and a decrease in the amplitude or even disappearance of the P wave. (The QRS complex being a combination of three of the graphical deflections seen on a typical electrocardiogram signal.)

On the other hand, in hypokalemia (a decreased blood potassium level), the T-wave tends to become flattened or even inverted. Additionally, there might be the presence of U waves, which are secondary waves following the T-wave, and a prolonged QT interval.

Both hyperkalemia and hypokalemia have profound clinical implications, and their respective ECG changes can provide invaluable clues to their presence, necessitating appropriate and timely interventions.

While these cardiac signals can provide indirect information about potassium levels, they are not always accurate and can be influenced by other physiological factors.

Thus, there exists a need for a more accurate system for estimating potassium levels in the human body.

The present disclosure proposes a system and method for measuring potassium levels through the fusion of data from a chemical sensor and a cardiac sensor.

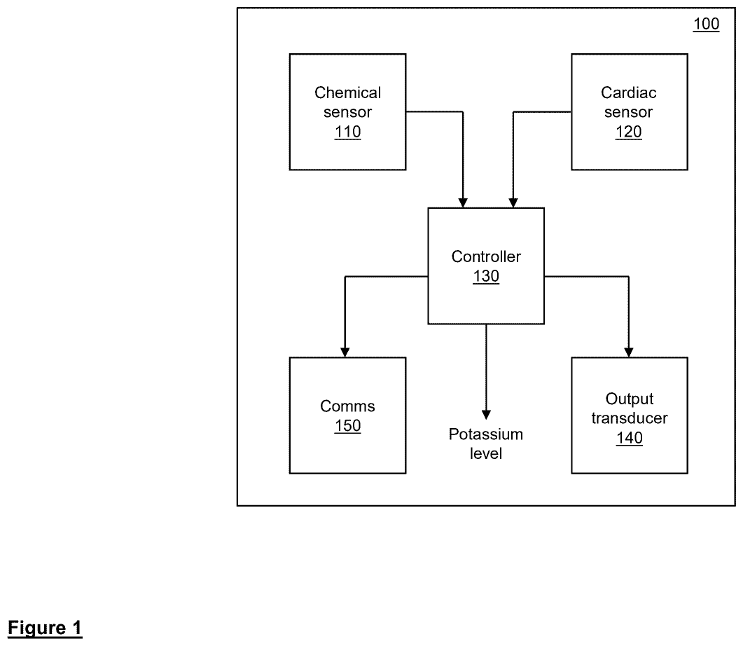

FIG. 1 is a schematic representation of an example system for potassium measurement according to the present disclosure.

The system, shown generally at 100 in FIG. 1, comprises a chemical sensor 110, a cardiac sensor 120, a controller, an output transducer subsystem 140 and a communications subsystem 150.

The chemical sensor 110 is configured to output a chemical sensor output signal indicative of a first estimate of a potassium level of user (e.g. a wearer) of the system 100. The chemical sensor 110 may comprise, for example, an ion-selective electrode (ISE) sensor. Alternatively, the chemical sensor 110 may comprise an aptamer-based sensor. Such a sensor may be configured to track a biomarker associated with potassium levels. Creatinine is an example of such a biomarker.

Ion-selective electrodes sensors (ISEs) are sensors that specifically measure the activity of a particular ion in an aqueous solution. This activity is usually related to the concentration of the ion. ISE sensors function based on a potential difference developed across a membrane, due to the difference in activity (or concentration) of the ion on either side of the membrane.

The relationship between the potential of the electrode (E) and the ion activity (a) is given by the Nernst equation. The Nernst equation describes how the potential difference across the electrode is proportional to the natural logarithm (or logarithm to base 10, in some formulations) of the ion activity. Specifically, it states that:

$$E = {E}_{0} + ( RT / nF ) ln ( a ) ,$$

where E₀ is the standard electrode potential, R is the universal gas constant, T is the absolute temperature, n is the charge number of the ion, and F is the Faraday constant. The Nernst equation provides a fundamental link between the thermodynamics of a system and its electrochemical properties, enabling the prediction and interpretation of the potentials observed in electroanalytical measurements.

In a two-terminal ISE sensor, the potentiometric measurement is obtained by measuring the potential difference between the ion-selective membrane of the ISE and a reference electrode when no current flows between them. The potential difference is directly related to the logarithm of the ion activity (or concentration) in the solution, as described by the Nernst equation.

A three-terminal ISE incorporates a working electrode, a reference electrode, and a counter electrode. This arrangement allows for potentiostatic measurements where the potential of the working electrode is controlled and maintained at a specific value relative to the reference electrode, and any resulting current (due to redox reactions at the working electrode) is passed through the counter electrode. While potentiometric measurements only measure potential difference at zero current, potentiostatic methods involve controlling the electrode's potential and measuring the resulting current, offering a different approach to analyse ion concentrations or other electrochemical properties. Typically, an ISE on a wearable device will be two electrode, whereas an ISE used with a fingerprick device will be three electrode.

The chemical sensor 110 may be implemented in a variety of different forms. For example, the chemical sensor 110 may comprise a wearable sensor configured to provide an output signal indicative of the first estimate of the user's potassium level. Alternatively, the chemical sensor 110 may comprise a sensor configured to analyse a sample of venous blood to generate the output signal indicative of the first estimate of the user's potassium level, or a sensor configured to analyse a sample of blood obtained from a fingerprick to generate the output signal indicative of the first estimate of the user's potassium level.

The cardiac sensor 120 is configured to output a cardiac sensor output signal indicative of a second estimate of the potassium level of the user. The cardiac sensor 120 may comprise, for example, an electrocardiogram (ECG) sensor or a photoplethysmogram (PPG) sensor.

An ECG sensor has one or more electrodes which, in use, are placed on the skin of the user to permit detection by the ECG sensor of electrical activity of the heart. Certain characteristics of the ECG waveform, like the T-wave, are known to be affected by potassium levels, as discussed above.

A PPG sensor is an optical sensor that measures blood volume changes in microvascular tissues. The shape, amplitude, and other attributes of the PPG waveform can provide insights into potassium levels.

Further detail on how the output from a heart sensor such as an ECG can be used to determine potassium level of a subject can be found in the paper “ECG frequency changes in Potassium disorders: a narrative review”, Teymouri et al., Am J Cardiovasc Dis 2022; 12 (3): 112-124; www.AJCD.us/ISSN:2160-200X/AJCD0142944, the contents of which are incorporated by reference herein.

The cardiac sensor 120 may be a wearable sensor. For example, a cardiac sensor 120 comprising an ECG sensor may be mounted on a chest strap that can be worn around the chest of the user, the chest strap having electrodes which are in contact with the user's skin when the chest strap is worn by the user, such that the ECG sensor can detect electrical activity of the user's heart.

As another example, a cardiac sensor 120 comprising a PPG sensor may be mounted on a wrist strap that can be worn on a wrist of the user, such that the PPG sensor is positioned in proximity to blood vessels in the user's wrist.

Outputs of the chemical sensor 110 and the cardiac sensor 120 are coupled to respective inputs of the controller 130, such that the controller 130 receives the chemical sensor output signal and the cardiac sensor output signal. The controller 130 is configured to execute one or more fusion algorithms to combine or fuse the first and second estimates of the user's potassium level to generate an output indicative of the user's potassium level.

Sensor fusion is a multidisciplinary methodology that combines data from multiple sensors to achieve more accurate and reliable information than would be possible when these sensors operate independently. By combining and processing information from different sources, sensor fusion mitigates the individual limitations and errors of each sensor. For instance, one sensor might be excellent at detecting an event under certain conditions but may falter under others, while a different sensor might have strengths in the areas where the first sensor is weak. When their data is combined, inconsistencies and inaccuracies can be identified and corrected. Moreover, the redundancy offered by multiple sensors can enhance system reliability. By employing algorithms that intelligently combine or integrate this multitude of data—often in real-time—sensor fusion provides a comprehensive, cohesive, and more confident representation of the monitored environment or system. This is especially critical in medical applications.

In medical testing there are two metrics used to score how well a system works:
i) sensitivity. This refers to the ability of a test to correctly identify those with the disease (true positive rate). In other words, among those who truly have the disease, how many did the test accurately detect? For example, if a test has 95% sensitivity, it means that it will correctly detect 95% of the people with the disease, but it will miss 5% of the people who have the disease.
ii) specificity. This refers to the ability of a test to correctly identify those without the disease (true negative rate). In other words, among those who do not have the disease, how many did the test accurately say were disease-free? For example, if a test has 90% specificity, it means that it will correctly identify 90% of the people without the disease, but will falsely indicate that 10% of the people without the disease have it.

In summary, sensitivity is a measure of how well the test identifies those with the disease, while specificity tells is a measure of how well the test identifies those without it.

Ideally, a medical test should have high values for both sensitivity and selectivity, but often there is a trade-off. The higher these metrics, the better the system's overall accuracy, as it implies a lower rate of both false acceptances and false rejections.

Sensor fusion can play a pivotal role in improving the accuracy of a medical test. By combining data from multiple sensors or sources, sensor fusion can compensate for the shortcomings of individual sensors. Each sensor might have unique vulnerabilities or error tendencies under certain conditions. When their outputs are intelligently combined or integrated, the strengths of one sensor can offset the weaknesses of another, thereby refining the decision-making process. This collective approach provided by sensor fusion can result in a more accurate and robust system.

In a medical system, the implications of a low sensitivity and low specificity can be dire. A low sensitivity system indicates that a test might not catch a lot of the true positive cases. So, for example, more individuals with a disease could receive negative test results, leading to a higher rate of false negatives (i.e. where the system erroneously rejects a diagnosis), which could lead to potentially life-threatening conditions being overlooked.

Conversely, a low specificity indicates that a test might produce many false positive results. In other words, it might indicate that individuals without the disease actually have it, which could lead to patients receiving unnecessary treatments or interventions. This not only exposes patients to undue risks associated with those treatments or interventions, but also results in wasted medical resources and increased costs.

The controller 130 is configured to generate an output indicative of the likely potassium level of a subject based on the combination or fusion of the chemical sensor output of the chemical sensor 110 and the cardiac sensor output of the cardiac sensor 130 heart sensor. This output may be referred to as an indicator.

The output transducer subsystem 140 may comprise one or more output transducers, for example an audio transducer such as a loudspeaker and/or a haptic transducer such as a vibrational actuator. The output transducer subsystem 140 is operable to generate an output for a user of the system 100 in response to a control signal output by the controller 130 if it is determined that the indicator of the potassium level indicates that the potassium level requires attention from the user. For example, the indicator of the potassium level may be compared (e.g. by the controller 130) to one or more threshold levels to identify a dangerously low or high level of potassium in the subject. If a dangerous level of potassium is detected, one or more of the output transducers of the output transducer subsystem 140 may be driven to generate an alert signal for a user of the system.

In some examples, the system 100 may further include (e.g. as part of the output transducer subsystem 150) a visual indicator such as a display for displaying data based on the indicator of the likely potassium level generated by the controller 130.

Additionally or alternatively, the system 100 may interface with a second device, external to the system 100, such as a smartphone, tablet or laptop computer, or wearable device such as a smart watch, smart glasses, a virtual reality (VR) or augmented reality (AR) headset or the like, to display the information relating to the monitored data (e.g. a numerical or graphical representation of the user's likely potassium level, based on the indicator generated by the controller 130) to the user.

To this end, the communications subsystem is configured to communicate, via a wired or wireless connection, with the external second device, to communicate data and/or alerts from the system 100 to the external device for notification and/or display to the user of the system 100. The communications subsystem 150 may be configured to communicate with the external device using a wireless connection such as Bluetooth®, Wi-Fi, near field communications (NFC), radio frequency identification (RFID) or the like. Additionally or alternatively, the communications subsystem 150 may be configured to communicate with the external device using a wired connection such as universal serial bus (USB).

In one example, the controller 130 may be configured to output the indicator to the communications subsystem 150 and to cause the communications subsystem 150 to transmit the indicator to the second, external, device for further processing by the second, external device, e.g. to generate a display and/or alert or notification to be output by the second, external device.

In some examples, the system 100 may comprise a predictor arranged to predict a level of potassium based on the cardiac sensor output signal output by the cardiac sensor 110.

The predictor may be a standalone element of the system 100, or alternatively may be provided as part of the controller 130. The predictor may comprise, for example, a trained neural network, artificial intelligence (AI) or other machine learning processor trained using suitable training data representing or indicative of the relationship between a potassium level and cardiac data such as ECG waves (e.g. changes to ECG waves due to changes in potassium levels).

The predictor may be configured to generate an estimate of a current and/or future potassium level of the user of the system 100 based on the cardiac sensor output signal output by the cardiac sensor 110.

In some examples, the predictor may have been further trained using suitable training data based on the output of a chemical sensor such as the chemical sensor 110 for different levels of potassium. In such examples, the predictor may receive the chemical sensor output signal output by the chemical sensor 120 as a further input, and may be configured to generate a more accurate estimate of the current and/or future potassium level based on the outputs from both the cardiac sensor 110 and the chemical sensor 120.

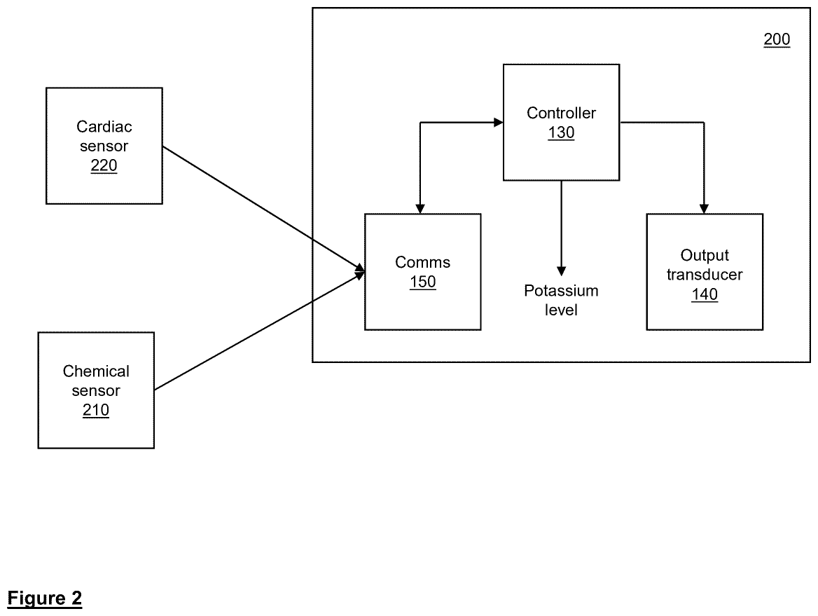

FIG. 2 is a schematic representation of an alternative example system for potassium measurement according to the present disclosure.

The system, shown generally at 200 in FIG. 2, has many features in common with the system 100 of FIG. 1. Such common features are denoted by common reference numerals in FIGS. 1 and 2, and will not be described again here, for the sake of clarity and brevity.

The system 200 differs from the system 100 in that it does not include an internal chemical sensor or an internal cardiac sensor. Instead, the system 200 receives the chemical sensor output signal from a chemical sensor 210 that is external to the system 200, and receives the cardiac sensor output signal from a cardiac sensor 220 that is external to the system 200. The chemical sensor output signal is received via a wireless or wired connection between the external chemical sensor 210 and the communications subsystem 150 of the system 200. Similarly, the cardiac sensor output signal is received via a wireless or wired connection between the external cardiac sensor 220 and the communications subsystem 150 of the system 200.

The external chemical sensor 210 may comprise, for example, an ISE sensor configured to generate a chemical sensor output signal indicative of a first estimate of a potassium level of a user.

The external cardiac sensor 220 may comprise, for example, an ECG-based sensor device such as a heart rate monitor, or may alternatively comprise a PPG-based cardiac sensor, which may be a standalone device or may be part of a host device such as a smartwatch or the like.

The controller 130 of the system 200 is operative in the same manner as the controller 130 described above to generate an output indicative of the (likely) potassium level of the user based on the first and second estimates based on the chemical sensor output signal received from the external chemical sensor 210 and from the external cardiac sensor 220, respectively.

The systems described above with reference to FIGS. 1 and 2 may be implemented, at least in part, in integrated circuits.

For example, the controller 130 and communications subsystem 140 of the systems 100, 200 of FIGS. 1 and 2 may be implemented in a single integrated circuit (IC), while the chemical sensor 110, cardiac sensor 120 and output transducer subsystem 140 may be external to the IC and coupled to suitable coupling terminals (e.g. balls, pads or pins) of the IC.

As will be appreciated from the foregoing disclosure, the systems disclosed herein provides an improved system for estimating or measuring a potassium level of a subject, which permits improved accuracy in potassium level measurements or estimates. This improved accuracy enables earlier recognition of problematic or potentially dangerous potassium levels and therefore earlier clinical interventions to investigate and rectify such potassium levels.

The skilled person will recognise that some aspects of the above-described apparatus and methods may be embodied as processor control code, for example on a non-volatile carrier medium such as a disk, CD- or DVD-ROM, programmed memory such as read only memory (Firmware), or on a data carrier such as an optical or electrical signal carrier. For many applications embodiments of the invention will be implemented on a DSP (Digital Signal Processor), ASIC (Application Specific Integrated Circuit) or FPGA (Field Programmable Gate Array). Thus the code may comprise conventional program code or microcode or, for example code for setting up or controlling an ASIC or FPGA. The code may also comprise code for dynamically configuring re-configurable apparatus such as re-programmable logic gate arrays. Similarly the code may comprise code for a hardware description language such as Verilog™ or VHDL (Very high speed integrated circuit Hardware Description Language). As the skilled person will appreciate, the code may be distributed between a plurality of coupled components in communication with one another. Where appropriate, the embodiments may also be implemented using code running on a field-(re) programmable analogue array or similar device in order to configure analogue hardware.

Note that as used herein the term module shall be used to refer to a functional unit or block which may be implemented at least partly by dedicated hardware components such as custom defined circuitry and/or at least partly be implemented by one or more software processors or appropriate code running on a suitable general purpose processor or the like. A module may itself comprise other modules or functional units. A module may be provided by multiple components or sub-modules which need not be co-located and could be provided on different integrated circuits and/or running on different processors.

As used herein, when two or more elements are referred to as “coupled” to one another, such term indicates that such two or more elements are in electronic communication or mechanical communication, as applicable, whether connected indirectly or directly, with or without intervening elements.

This disclosure encompasses all changes, substitutions, variations, alterations, and modifications to the example embodiments herein that a person having ordinary skill in the art would comprehend. Similarly, where appropriate, the appended claims encompass all changes, substitutions, variations, alterations, and modifications to the example embodiments herein that a person having ordinary skill in the art would comprehend. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative. Accordingly, modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

Although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described above.

Unless otherwise specifically noted, articles depicted in the drawings are not necessarily drawn to scale.

All examples and conditional language recited herein are intended for pedagogical objects to aid the reader in understanding the disclosure and the concepts contributed by the inventor to furthering the art, and are construed as being without limitation to such specifically recited examples and conditions. Although embodiments of the present disclosure have been described in detail, it should be understood that various changes, substitutions, and alterations could be made hereto without departing from the spirit and scope of the disclosure.

Although specific advantages have been enumerated above, various embodiments may include some, none, or all of the enumerated advantages. Additionally, other technical advantages may become readily apparent to one of ordinary skill in the art after review of the foregoing figures and description.

It should be noted that the above-mentioned embodiments illustrate rather than limit the invention, and that those skilled in the art will be able to design many alternative embodiments without departing from the scope of the appended claims. The word “comprising” does not exclude the presence of elements or steps other than those listed in a claim, “a” or “an” does not exclude a plurality, and a single feature or other unit may fulfil the functions of several units recited in the claims. Any reference numerals or labels in the claims shall not be construed so as to limit their scope.

1. A system for potassium measurement comprising:
a chemical sensor configured to output a chemical sensor output signal indicative of a first estimate of a potassium level of a user of the system;
a cardiac sensor configured to output a cardiac sensor output signal indicative of a second estimate of the potassium level of the user; and
a controller configured to generate an output indicative of the potassium level of the user based on the first and second estimates.

2. The system of claim 1, wherein the chemical sensor comprises an ion-selective electrode (ISE) sensor.

3. The system of claim 1, wherein the chemical sensor comprises a potentiometric sensor.

4. The system of claim 1, wherein the chemical sensor comprises a wearable sensor.

5. The system of claim 1, wherein the chemical sensor is configured to analyse a sample of venous blood or to analyse a sample of blood obtained from a fingerprick.

6. The system of claim 1, wherein the chemical sensor comprises an aptamer-based sensor configured to tracks a biomarker associated with potassium levels.

7. The system of claim 1, wherein the cardiac sensor comprises an electrocardiogram (ECG) sensor.

8. The system of claim 1, wherein the cardiac sensor comprises a photoplethysmogram (PPG) sensor.

9. The system of claim 1, further comprising an output transducer subsystem.

10. The system of claim 9, wherein the output transducer subsystem comprises one or more of:
an audio output transducer;
a haptic output transducer; and
a visual indicator.

11. The system of claim 1, further comprising a communications subsystem configured to communicate data and/or alerts from the system to an external device.

12. The system of claim 1, further comprising an output transducer subsystem and a communications subsystem, wherein the controller is configured to generate an output signal for one or more of output transducer subsystem and the communications subsystem based on the output indicative of the potassium level.

13. The system of claim 1, further comprising a predictor configured to generate an estimate of a current and/or future potassium level of the user based on the cardiac sensor output signal.

14. The system of claim 13, wherein the predictor is configured to generate the estimate of the current and/or future potassium level of the user based on the cardiac sensor output signal and the chemical sensor output signal.

15. The system of claim 13, wherein the predictor comprises a trained neural network, artificial intelligence or other machine learning processor.

16. The system of claim 15, wherein the predictor has been trained on a relationship between a potassium level and cardiac data to generate, based on the cardiac sensor output signal, the estimate of the current and/or future potassium level of the user.

17. The system of claim 14, wherein the predictor comprises a trained neural network, artificial intelligence or other machine learning processor, wherein the predictor has been trained on a relationship between a potassium level and cardiac data and for different levels of potassium to generate, based on the cardiac sensor output signal and the chemical sensor output signal, the estimate of the current and/or future potassium level of the user

18. An integrated circuit comprising a controller configured to:
receive, from a chemical sensor, a chemical sensor output signal indicative of a first estimate of a potassium level of a user of the system;
receive, from a cardiac sensor, a cardiac sensor output signal indicative of a second estimate of the potassium level of the user; and
generate an output indicative of the potassium level of the user based on the first and second estimates.

19. A method for measuring a potassium level of a subject, the method comprising:
generating a first estimate of the potassium level using a chemical sensor;
generating a second estimate of the potassium level using a cardiac sensor; and
generating an indication of the potassium level based on a fusion of the first and second estimates.

20. A system for potassium measurement comprising:
a controller configured to:
receive, from a chemical sensor, a chemical sensor output signal indicative of a first estimate of a potassium level of a user of the system;
receive, from a cardiac sensor, a cardiac sensor output signal indicative of a second estimate of the potassium level of the user; and
generate an output indicative of the potassium level of the user based on the first and second estimates.
